(19) United States
(12) Patent Application Publication
(10) Pub. No.: US 2012/0157370 A1
(43) Pub. Date: Jun. 21, 2012

(54) WASHING PERFORMANCE USING RADICAL TRAPS

(75) Inventors: Christian Kropf, Hilden (DE); Andreas Buhl, Langenfeld (DE); André Hätzelt, Dusseldorf (DE)

(73) Assignee: Henkel AG & Co. KGaA, Dusseldorf (DE)

(21) Appl. No.: 13/403,197

(22) Filed: Feb. 23, 2012

Related U.S. Application Data
(63) Continuation of application No. PCT/EP2010/062375, filed on Aug. 25, 2010.

(30) Foreign Application Priority Data
Aug. 26, 2009 (DE) 10 2009 028 891.0

(51) Int. Cl. C11D 3/20; C07D 311/72; C07F 9/10; C07D 307/62; C07F 9/655; C07C 55/14; C07C 59/255; C07C 57/10; C07C 69/704; C07C 69/84; C07C 59/265

(52) U.S. Cl. 510/276; 560/70; 549/408; 558/169; 549/315; 562/584; 562/590; 562/585; 562/601; 560/180; 549/317; 549/222

(57) ABSTRACT

The invention relates to the use of radical traps in detergents and cleaners in order to improve cleaning performance, in particular for dirt containing polymerizable dyes.

### CROSS-REFERENCES TO RELATED APPLICATIONS

This application is a continuation of PCT/EP2010/062375, filed on Aug. 25, 2010, which claims priority under 35 U.S.C. §119 to DE 10 2009 028 891.0 filed on Aug. 26, 2009, both of which are hereby incorporated by reference.

### FIELD OF THE INVENTION

The present invention generally relates to the use of radical traps in washing and cleaning agents to improve cleaning performance, in particular with respect to stains that contain polymerizable dyes.

### BACKGROUND OF THE INVENTION

While the formulation of powdered, bleach-containing washing and cleaning agents no longer presents any problems today, the formulation of stable liquid, bleach-containing washing and cleaning agents continues to represent a problem.

Because of the absence of the bleaching agent, particularly in liquid washing and cleaning agents, those stains that are normally removed particularly because of the bleaching agents contained are often, accordingly, removed only in an inadequate manner.

A similar problem also exists for bleach-free color-washing agents, in which the bleaching agent is omitted in order to protect the dyes in the textile and to prevent them from being bleached.

In the absence of the bleaching agent, an added problem is that often, instead of stains that are normally removed by the bleaching agent being removed, on the contrary, the stain is even intensified or made more difficult to remove as a result of the washing process, not least because of chemical reactions that are initiated, which can, for example, consist in the polymerization of certain dyes that are contained in the stains.

These problems occur in particular with stains that contain polymerizable dyes. These are mainly red- to blue-colored stains. The polymerizable substances are especially polyphenolic dyes, preferably flavonoids, in particular from the class of the anthocyanidins or anthocyanins. The stains can, in particular, be red wine stains or stains of fruit or vegetables which contain red and/or blue dyes, in particular polyphenolic dyes, especially those from the class of the anthocyanidins or anthocyanins. The stains can, in particular, have been caused by food products or drinks that contain corresponding dyes.

According to the invention, it has now surprisingly been found that, by adding radical traps to those washing and cleaning agents, the cleaning performance of the washing or cleaning agent can be significantly improved with respect to those stains.

The use of radical traps in washing and cleaning agents is already described in the prior art. However, the use of radical traps described in the prior art serves to stabilize components that are contained, which can be inactivated and/or destroyed by oxidative degradation.

Thus, in EP0209228, EP0668345, EP0843001, EP1001010 and EP1462564, bleaching agents are disclosed which contain radical traps to stabilize “bleach-unstable” components.

In EP1144580, the use of radical traps in transparent packaging to prevent damage by UV light to components that are contained is described.

In WO2006/128554 too, the use of radical traps to protect the components that are contained from oxidative degradation is disclosed.

However, the use of radical traps in washing and cleaning agents to improve the cleaning performance has not yet been described in the prior art.

The present invention therefore firstly provides the use of radical traps in washing and cleaning agents to improve cleaning performance.

Furthermore, other desirable features and characteristics of the present invention will become apparent from the subsequent detailed description of the invention and the appended claims, taken in conjunction with this background of the invention.

### BRIEF SUMMARY OF THE INVENTION

Use of radical traps in washing and cleaning agents for improving the cleaning performance. A textile-washing agent containing a compound of general formula (I)

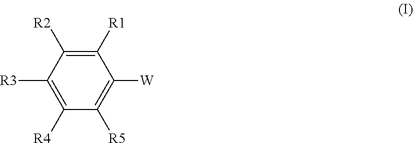

wherein W denotes a residue—C(O)X or —NLC(O)X, X denotes a residue R, OR or NRR′, L denotes H or C₁₋₆ alkyl, R and R′, independently of one another, denote H or a linear or branched C₁₋₄₀ alkyl residue, in particular a C₄₋₂₀ alkyl residue, especially a C₆₋₁₄ alkyl residue, wherein the alkyl residue may optionally also be mono- or polysubstituted, in particular by residues selected from hydroxy and C₁₋₆ alkoxy, and/or can contain one or more hetero atoms, in particular selected from O, N and S, R1, R2, R3, R4 and R5, independently of one another, denote H, hydroxy, C₁₋₆ alkyl, in particular methyl or ethyl, or C₁₋₆ alkoxy, in particular methoxy or ethoxy.

A textile-washing agent, containing ascorbyl phosphate and/or a salt thereof.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In particular, the present invention provides the use of radical traps in washing and cleaning agents for the improved removal of stains that contain polymerizable substances, in particular polymerizable dyes, the polymerizable dyes preferably being polyphenolic dyes, in particular flavonoids, especially anthocyanidins or anthocyanins or oligomers of these compounds. The stains are preferably red- to blue-colored stains, in particular red wine stains or stains of fruit or vegetables that contain red- to blue-colored dyes, and in particular also stains from food products or drinks that contain corresponding dyes.

The term “red- to blue-colored stains” is to be understood according to the invention as stains that can have a color from the color spectrum of red to blue. In addition to stains in the colors red or blue, therefore, in particular stains in intermediate colors, in particular in violet, lilac, purple or pink, are also included as well as stains that have a red, violet, lilac, purple, pink or blue shade, without substantially themselves having to consist completely of this color. The above-mentioned colors can, in particular, also be light or dark in each case, i.e. in particular light and dark red and light and dark blue are included as possible colors. The stains to be removed according to the invention can in particular be caused by cherries, red grapes, pomegranate, chokeberries, plums, sea buckthorn, açai, berries, especially red or blackcurrants, elderberries, blackberries, raspberries, blueberries, cranberries, strawberries or bilberries, red cabbage, blood orange, eggplant, black carrots, red-fleshed or blue-fleshed potatoes or red onions.

### Radical Traps

The radical traps can be any compound that is capable of trapping radicals and, as a result, stopping a radical chain reaction.

In a preferred embodiment, the radical trap is an aromatic compound or a compound that comprises aromatic residues. The aromatic compound or the aromatic residues can, in particular, be optionally substituted benzene, naphthalene, annulene, cyclopentadiene, cyclopropene, anthracene or phenanthrene, with benzene and naphthalene being particularly preferred.

The substituents here are preferably selected from alkyl, in particular C₁₋₆ alkyl, hydroxy, alkoxy, in particular C₁₋₆ alkoxy, amino, alkylamino, in particular C₁₋₆ alkylamino, dialkylamino, in particular di-(C₁₋₆ alkyl)amino, amido, alkylamido, in particular C₁₋₆ alkylamido, dialkylamido, in particular di-(C₁₋₆ alkyl)amido, halogen, in particular fluorine, chlorine or bromine, hydrogen carbonyl, alkyl carbonyl, in particular acetyl, alkoxycarbonyl, in particular C₁₋₆ alkoxycarbonyl, oligooxyethylene, in particular—(O—CH₂—CH₂—)ₙOH with n=1 to 20, especially 1 to 10, allyl, nitro, carboxy and sulfo.

Particularly preferred here are those aromatic compounds or aromatic residues that have one hydroxyl group, two hydroxyl groups, three hydroxyl groups, one methoxy group, two methoxy groups, three methoxy groups, a sulfonic acid group, a carboxylic acid group or a carboxylic acid ester group—optionally in addition to others of the above-mentioned substituents in each case.

The radical trap according to the invention is preferably selected here from mono-, di- and trihydroxybenzenes, in particular mono-tert-butylhydroxytoluene, di-tert-butylhydroxytoluene, p-hydroxytoluene, hydroquinone, mono-tert-butylhydroquinone, di-tert-butylhydroquinone, allylhydroquinone, acetylhydroquinone, pyrocatechol, mono-tert-butyl pyrocatechol, di-tert-butyl pyrocatechol, allylpyrocatechol, acetylpyrocatechol, p-hydroxyanisole, tert-butylhydroxyanisole, tert-butyl hydroxyaniline, p-hydroxyaniline, methylphenol, ethylphenol, methoxyphenol, ethoxyphenol, methoxypropenylphenol, dihydroxybenzaldehyde, gallic acid and methyl, ethyl, propyl, hexyl, octyl or dodecyl gallic acid, benzylamine, diphenylamine, aryl carboxylates, in particular optionally substituted benzoic acid, especially hydroxybenzoic acid, dihydroxybenzoic acid, trihydroxybenzoic acid, methoxyhydroxybenzoic acid, methoxydihydroxybenzoic acid, dimethoxyhydroxybenzoic acid, dihydroxyterephthalic acid, methylbenzoic acid, dimethoxybenzoic acid, trimethoxybenzoic acid, tetramethoxybenzoic acid or pentamethoxybenzoic acid, or an ester or salt thereof, optionally substituted benzenesulfonic acid or a salt of this compound.

In a particularly preferred embodiment according to the invention, a compound of general formula (I)

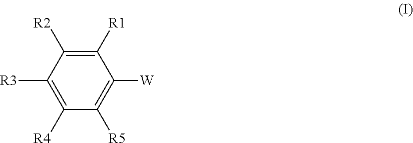

is used as the radical trap, wherein
W denotes a residue —C(O)X or —NLC(O)X,
X denotes a residue R, OR or NRR′,
L denotes H or C₁₋₆ alkyl,
R and R′, independently of one another, denote H or a linear or branched C₁₋₄₀ alkyl residue, in particular C₄₋₂₀ alkyl residue, especially C₆₋₁₄ alkyl residue, wherein the alkyl residue may optionally also be mono- or polysubstituted, in particular by residues selected from hydroxy and C₁₋₆ alkoxy, and/or can contain one or more hetero atoms, in particular selected from O, N and S,
R1, R2, R3, R4 and R5, independently of one another, denote H, hydroxy, C₁₋₆ alkyl, in particular methyl or ethyl, or C₁₋₆ alkoxy, in particular methoxy or ethoxy.

Particularly preferably, a compound of general formula (II)

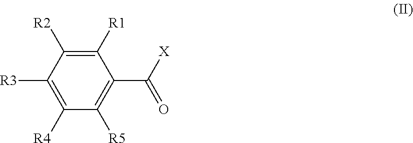

or of general formula (III)

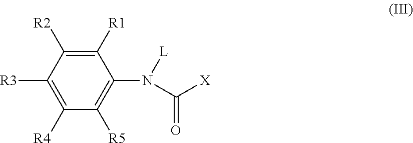

is used as the radical trap,
wherein in formula (II)
X denotes a residue OR or NRR′,
R and R′, independently of one another, denote H or a linear or branched C₁₋₄₀ alkyl residue, in particular C₄₋₂₀ alkyl residue, especially C₆₋₁₄ alkyl residue, wherein the alkyl residue may optionally also be mono- or polysubstituted, in particular by residues selected from hydroxy and C₁₋₆ alkoxy, and/or can contain one or more hetero atoms, in particular selected from O, N and S,
R2, R3, R4 and R5, independently of one another, denote H, hydroxy, C₁₋₆ alkyl, in particular methyl or ethyl, or C₁₋₆ alkoxy, in particular methoxy or ethoxy;
and in formula (III)
X denotes a residue R or OR,
L denotes H or C₁₋₆ alkyl,
R denotes H or a linear or branched C₁₋₄₀ alkyl residue, in particular C₄₋₂₀ alkyl residue, especially C₆₋₁₄ alkyl residue, wherein the alkyl residue may optionally also be mono- or polysubstituted, in particular by residues selected from hydroxy and C₁₋₆ alkoxy, and/or can contain one or more hetero atoms, in particular selected from O, N and S,
R1, R2, R3, R4 and R5, independently of one another, denote H, hydroxy, C₁₋₆ alkyl, in particular methyl or ethyl, or C₁₋₆ alkoxy, in particular methoxy or ethoxy.

The above-mentioned alkyl residue containing hetero atoms is preferably a residue

—(CH₂—)ₘ(CH₂—CH₂—O—)ₙ(CH₂—)ₒZ,

wherein
m and o, independently of one another, denote a number from 0 to 10,
n denotes a number from 1 to 20,
with the proviso that m+2n+o is preferably less than or equal to 40, in particular less than or equal to 20,
Z denotes hydrogen or—for o 1—hydroxy or C₁₋₆ alkoxy, in particular methoxy or ethoxy.

In a preferred embodiment, the residue X in each case denotes OR and at least one the residues R2, R3 and R4, preferably at least the residue R3, in each case denotes hydroxy.

In a particularly preferred embodiment,

X denotes OR,
R denotes a linear or branched C₁₋₄₀ alkyl residue, in particular C₄₋₂₀ alkyl residue, especially C₆₋₁₄ alkyl residue,
R1 and R5 denote hydrogen,
R2, R3 and R4 denote hydroxy.

Particularly preferably, R denotes in each case a linear or branched residue selected from hexyl, heptyl, octyl, nonyl, decyl, undecyl, dodecyl, tridecyl and tetradecyl.

The compound which can comprise the above-mentioned aromatic compounds as residues can in particular be a polymer which contains these residues in the backbone and/or in the side chain. Examples of these are in particular polystyrene, copolymers of styrene and other compounds, in particular of styrene and maleic acid and/or acrylic acid, graft polymers of styrene and other compounds, in particular of styrene and ethylene glycol, polyethylene glycol ditoluenesulfonate, polyhydroxystyrene, polymethylstyrene, polystyrene divinylbenzene and polyvinylphenol.

In another preferred embodiment, the radical trap is a sterically and/or cyclically hindered amine. This can in particular be a 2,2,6,6-tetraalkylpiperidine, in particular a 2,2,6,6-tetramethylpiperidine, which may optionally carry other substituents, in particular selected from C₁₋₆ alkyl, hydroxy, alkoxy, in particular C₁₋₆ alkoxy, amino, alkylamino, in particular C₁₋₆ alkylamino, dialkylamino, in particular di-(C₁₋₆ alkyl)amino, halogen, in particular fluorine, chlorine or bromine, hydrogen carbonyl, alkyl carbonyl, in particular acetyl, allyl, nitro, carboxy and sulfo. In addition, the nitrogen atom of the piperidine backbone may optionally also be substituted, in particular by alkyl, in particular methyl or ethyl, oxy, hydroxy or alkoxy, in particular methoxy or ethoxy.

Also suitable as radical traps are alkyl carboxylates, tocopherol, lecithin, thiodipropionate, organic acids, in particular ascorbic acid, citric acid, adipic acid, tartaric acid or sorbic acid and derivatives of these compounds, in particular triethyl citrate, ascorbyl palmitate, ascorbyl stearate, ascorbyl glucoside or ascorbyl sulfate; moreover, amino acids, in particular glutamine, methionine or cysteine, are also suitable as radical traps.

With regard to radical traps that can be used according to the invention, reference is also made in particular to the patent application US2007/0287651.

The radical trap is used according to the invention preferably in a quantity of 0.001 to 10 wt. %, in particular in a quantity of 0.01 to 5 wt. %, particularly preferably in a quantity of 0.05 to 2 wt. %.

The washing or cleaning agent here can be present in any form of administration that is established according to the prior art and/or useful. These include, for example, solid, powdered, liquid, gel or paste forms of administration, optionally also comprising several phases, compressed or non-compressed; they also include, for example: extrudates, granules, tablets or pouches, both in bulk containers and packaged in individual doses.

The use according to the invention takes place in a preferred embodiment according to the invention in a washing or cleaning agent that contains no bleaching agents. This is preferably understood according to the invention to mean that, as well as bleaching agents in the narrower sense, i.e. as well as hydrogen peroxide or substances providing hydrogen peroxide, the agent according to the invention also contains no bleach activators and/or bleach catalysts.

The washing or cleaning agent according to the invention in a particularly preferred embodiment is a liquid textile-washing agent.

The washing or cleaning agent according to the invention in another particularly preferred embodiment is a powdered color-washing agent, i.e. a powdered textile-washing agent for colored textiles.

The use of the above-mentioned compounds of general formula (I), in particular of general formula (II) and of general formula (III), has proved to be particularly preferred according to the invention for enhancing cleaning performance, the special embodiments mentioned as being preferred in each case having proved particularly advantageous.

The present invention therefore also provides a textile-washing agent containing the above-mentioned compounds of general formula (I). In particular, it preferably provides a textile-washing agent containing compounds of general formula (II) and a textile-washing agent containing compounds of general formula (III).

The use of ascorbyl phosphate and/or its salts has also proved to be particularly preferred according to the invention for enhancing cleaning performance.

The present invention therefore also provides a textile-washing agent containing ascorbyl phosphate and/or one of its salts.

The textile-washing agent according to the invention is preferably a liquid textile-washing agent and/or a textile-washing agent which is free from bleaching agents.

The textile-washing agents according to the invention and the washing and cleaning agents in which the use according to the invention takes place can additionally contain other conventional components of washing and cleaning agents, in particular of textile-washing agents, in particular selected from the group of the builders, surfactants, polymers, enzymes, fabric-softening substances, in particular esterquats, protein hydrolyzates, electrolytes, pH regulators, fluorescent agents, hydrotopes, foam inhibitors, silicone oils, antiredeposition agents, optical brighteners, graying inhibitors, shrinkage preventers, anti-crease agents, dye transfer inhibitors, active antimicrobial substances, germicides, fungicides, antioxidants, antistatic agents, ironing aids, proofing and impregnating agents, swelling and anti-slip agents, UV absorbers, disintegrants, perfumes, dyes and perfume carriers.

As builders, in particular zeolites, silicates, carbonates, organic co-builders and/or also—where there are no environmental objections to their use—phosphates can be used according to the invention.

As surfactants, in particular nonionic, anionic, cationic and/or amphoteric surfactants can be used according to the invention.

As enzymes, in particular proteases, amylases, lipases, hemicellulases, cellulases, perhydrolases and/or oxidoreductases can be used according to the invention.

### EXEMPLARY EMBODIMENTS

### Example 1

### Use of Propyl Gallate (PG) to Improve Washing Performance

Washing tests were carried out on 5 different stains, which were based on polyphenolic natural dyes (flavonoids). For the stains, extracts of cherries, blackcurrants, bilberries, red grapes and red wine were used. The stain production took place mechanically by measuring out a constant quantity of a dilute aqueous solution of the extracts onto cotton fabric and then drying. For the washing tests, a commercial liquid washing agent (LWA) was used and washing was performed at 40° C. in a conventional textiles washing machine at 16° dH water hardness. Five determinations were carried out and then the average was determined in each case. 75 g of the washing agent was measured out in each case; propyl gallate (PG) was added on top in a quantity of 1.7 g. The evaluation took place by means of color distance measurement according to the Lab values and the Y values calculated therefrom as a measure of lightness. The following table shows the dY values, which are obtained from the difference Y (after washing)—Y (before washing), for the 5 above-mentioned stains.

| | Cherry | Blackcurrant | Bilberry | Red grape | Red wine |
|---|---|---|---|---|---|
| LWA | 35.7 | 50 | 27.6 | 52.8 | 40.6 |
| LWA + PG | 41.7 | 54 | 33.3 | 62 | 54.5 |

The dY values with the addition of PG are greater than with the pure LWA for all of the stains, which corresponds to a higher degree of whiteness and thus improved stain removal.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A textile-washing agent comprising a radical trap compound of general formula (I) 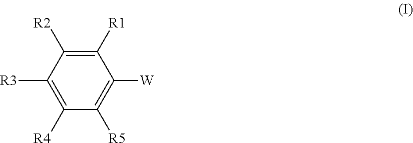
wherein:
a.) W denotes a residue —C(O)X or —NLC(O)X,
X denotes a residue R, OR or NRR′,
L denotes H or C₁₋₆ alkyl,
R and R′, independently of one another, denote H or a linear or branched C₁₋₄₀ alkyl residue, wherein the alkyl residue may optionally also be mono- or polysubstituted, and/or can contain one or more hetero atoms;
b.) R1, R2, R3, R4 and R5, independently of one another, denote H, hydroxy, C₁₋₆ alkyl.

2. The textile-washing agent according to claim 1 wherein the radical trap compound general formula (I) is defined as:
X denotes a residue OR;
R denotes a linear or branched C₁₋₄₀ alkyl residue;
R1 and R5 denote hydrogen;
R2, R3 and R4 denote hydroxy.

3. The textile-washing agent according to claim 1 wherein the radical trap is selected from mono- and di- and trihydroxybenzenes.

4. The textile-washing agent according to claim 1 wherein the radical trap is selected from mono-tert-butylhydroxytoluene, di-tert-butylhydroxytoluene, p-hydroxytoluene, hydroquinone, mono-tert-butylhydroquinone, di-tert-butylhydroquinone, allylhydroquinone, acetylhydroquinone, pyrocatechol, mono-tert-butyl pyrocatechol, di-tert-butyl pyrocatechol, allylpyrocatechol, acetylpyrocatechol, p-hydroxyanisole, tert-butyl hydroxyanisole, tert-butyl hydroxyaniline, p-hydroxyaniline, methylphenol, ethylphenol, methoxyphenol, ethoxyphenol, methoxy propenyl phenol, dihydroxybenzaldehyde, gallic acid and methyl, ethyl, propyl, hexyl, octyl or dodecyl gallic acid, benzylamine.

5. The textile-washing agent according to claim 1 wherein the radical trap is selected from hydroxybenzoic acid, dihydroxybenzoic acid, trihydroxybenzoic acid, methoxyhydroxybenzoic acid, methoxydihydroxybenzoic acid, dimethoxyhydroxybenzoic acid, dihydroxyterephthalic acid, methylbenzoic acid, dimethoxybenzoic acid, trimethoxybenzoic acid, tetramethoxybenzoic acid or pentamethoxybenzoic acid, or an ester or salt thereof.

6. The textile-washing agent according to claim 1 wherein the textile-washing agent contains no bleaching agents.

7. The textile-washing agent according to claim 6 wherein the radical trap compound comprises from between 0.001 to 10.0 wt. %.

8. A textile-washing agent comprising a radical trap selected from tocopherol, lecithin, thiodipropionate, ascorbic acid, citric acid, adipic acid, tartaric acid sorbic acid, triethyl citrate, ascorbyl palmitate, ascorbyl stearate, ascorbyl glucoside, ascorbyl sulfate, and ascorbyl phosphate.

9. The textile-washing agent according to claim 7 wherein the textile-washing agent contains no bleaching agents.

10. A method for washing textiles wherein a washing agent comprising a radical trap compound of general formula (I) 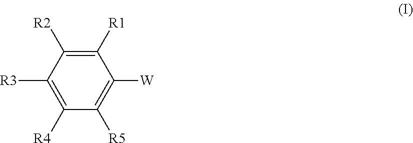
wherein:
a.) W denotes a residue —C(O)X or —NLC(O)X,
X denotes a residue R, OR or NRR′,
L denotes H or C₁₋₆ alkyl,
R and R′, independently of one another, denote H or a linear or branched C₁₋₄₀ alkyl residue, wherein the alkyl residue may optionally also be mono- or polysubstituted, and/or can contain one or more hetero atoms;
b.) R1, R2, R3, R4 and R5, independently of one another, denote H, hydroxy, C₁₋₆ alkyl;
is contacted with stained fabric in a conventional washing machine.
